(19) United States
(12) Patent Application Publication
(10) Pub. No.: US 2021/0304877 A1
(43) Pub. Date: Sep. 30, 2021

(54) SYSTEMS AND METHODS FOR ASSIGNING EXAMS TO PHYSICIANS

(71) Applicant: Change Healthcare Holdings, LLC, Nashville, TN (US)

(72) Inventors: Tomer Zonens, Tel-Aviv (IL); Uri Maimon, Shoham (IL); Hadar Dayan, Holon (IL); Omer Schalit-Cohen, Ramat-Gan (IL); Ran Rumianek, Vancouver (CA); Amir Wigelman, Kochav-Yair (IL); Amit Bezalel, Beer-Yaakov (IL)

(21) Appl. No.: 17/175,808

(22) Filed: Feb. 15, 2021

Related U.S. Application Data
(60) Provisional application No. 63/002,787, filed on Mar. 31, 2020.

(51) Int. Cl. G16H 40/20; G06Q 10/06; G06Q 10/04

(52) U.S. Cl. CPC G16H 40/20; G06Q 10/06393; G06Q 10/04; G06Q 10/067; G06Q 10/06316

(57) ABSTRACT

A system that actively matches supply (physicians, technicians, or other healthcare providers) with demand (exams) in hospitals, clinics and telemedicine environments is provided. The system may be used along with the hospital or other medical facility's imaging and information systems, which allows the system to be aware of any exam that needs to be interpreted by a healthcare provider (e.g., a CT scan that needs to be interpreted by a radiologist).

### CROSS-REFERENCE TO RELATED APPLICATIONS

This application claims priority to U.S. Provisional Patent Application Ser. No. 63/002,787, entitled SYSTEMS AND METHODS FOR ASSIGNING EXAMS TO PHYSICIANS, and filed on Mar. 31, 2020. The disclosure of which is incorporated by reference in its entirety.

### BACKGROUND

Managing physician workflows is a dynamic and difficult task. It becomes more challenging as the scale of a healthcare system grows, which is the trend in today's consolidating provider market. While hundreds of millions of exams are conducted on patients with different medical conditions, and with different levels of urgency that mandate different care pathways, most hospitals do not have the technology to prioritize, triage and assign them such that each exam is handled by the right physician at the right time. This failure is experienced even in low volume settings, so for example a low priority normal x-ray might be read before a critical brain CT scan of a patient in the emergency department.

As a result, multiple aspects of the workflow are sub-optimal. There are many inefficiencies, and patient care is not as good as it could be. Some examples of such inefficiencies include: exam turnaround times are too high; some exams “fall through the cracks”—ignored, de-prioritized, left for “someone else” to interpret (cherry-picking); workload imbalance—some physicians may be idle while others are overwhelmed; exams are assigned to physicians that are too busy to handle them in time; and assigning exams to someone other than the best fitting subspecialist because of lack of awareness to subspecialist availability.

### SUMMARY

An agile, goal-driven workflow orchestration system that actively matches supply (physicians, technicians or other healthcare providers) with demand (exams) in hospitals, clinics and telemedicine environments is provided. The system may be used along with the hospital or other medical facility's imaging and information systems, which allows the system to be aware of any exam that needs to be interpreted by a healthcare provider (e.g., a CT scan that needs to be interpreted by a radiologist).

In one embodiment, each new exam that arrives at the system may be analyzed and modeled. In parallel, the system may monitor the activity of logged-in providers (e.g., doctors). Each provider may also be modeled, and the model may be continuously improved as that provider interprets more exams.

Each change in the system state (inbound data, provider logging in/out, etc.) may trigger a calculation of the optimal assignment of available providers to available exams. The assignment may be based on multiple metrics, such as exam priority, exam SLA (Service Level Agreement), exam complexity, and/or provider location, skills, credentialing, preferences, etc.

According to one embodiment, an optimization engine may use the customer's (e.g., a healthcare organization) goals as input for the optimization process. The customer can modify the behavior of the system using simple controls that allow them to understand what impact these changes would have on the system. For example, one customer may put more emphasis on timeliness while another may emphasize quality of results. In one embodiment, providers using the system may be assigned one exam at a time, eliminating the need to select which exam to read next. Once the provider is done with the exam, the system may present the next one to them automatically.

The optimization engine may also consider one or more constraints in the optimization process. Example constraints may include exam or procedure quotas for the customer, or budgets for a customer. For example, each exam may be assigned a cost or dollar value. The total cost for a customer may be monitored to ensure that the total cost does not exceed a predetermined budget for a specified time period.

In one embodiment, the system flow may be described as follows: an exam may be entered into the system and the exam metadata may be modeled and enhanced to estimate various parameters including, for example, its priority, its difficulty to interpret, its required skills to interpret, and other factors. The system may further estimate the availability of each provider, and the exam may be put into an interpretation backlog (e.g., queue). The system may then calculate the optimal assignment of providers to exams that considers the availability of the providers and the exam metadata. Once a provider completes a current exam or task, the provider may receive a next assigned exam. The system may further use data about each exam completion (e.g., time to completion, the provider that completed exam, and feedback from the patient) to enrich the assignment model to improve future exam assignments. Each new exam, completed exam, data update and provider logging in or out of the system may trigger a recalculation of the assignments. Any change to the goal configuration may affect the assignment algorithm in real-time.

The systems and methods described herein provide the following advantages. With existing solutions, there is no direct link between goals and outcomes. As those solutions are based on static rules, they fail in front of the dynamic nature of workflow in healthcare systems. In addition, the complexity and large number of rules make them very fragile and hard to scale, as any change to the rules might result in unpredictable adverse outcomes. The new proposed solution actively solves the underlying optimization problem—on the one hand there is a continuous flux of exams performed, and on the other hand there are providers, each with their own set of skills, availability and so on. The described systems and methods may match exams (demand) with providers (supply) in an optimal way—optimal in a sense of meeting the goals set by the organization (i.e., customer). A difference in this new approach is treating the problem as a supply/demand problem that is solved by optimization techniques, while existing solutions try to mimic the manual management of the same problem. Another difference is the ability to configure the behavior of the system in a much more explicit and predictable way, by modifying the goals that will be used by the optimization engine. Finally, the proposed approach is dynamic and adapts in real-time to changes in the workflow environment such as changes in the availability of providers and changes in the number of new exams.

In another aspect of the invention, a system for simulating provider workflow is provided. The system may include several functionalities including recording workflow related events. These events may include exam metadata, properties and timing information; provider activity information and provider properties and/or metadata. The system may further export historical workflow information, including exam and provider data, feed the workflow system with exported workflow data, and run the workflow system in a “simulation mode.” In the simulation mode timing information is preserved but the simulation itself is run very quickly (e.g., the data exported may represent very long periods, but the simulation runs in minutes). The system may further generate simulation reports that include multiple KPIs (Key Performance Indicators) such as turnaround times for different exam types, productivity per provider, and more.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is an illustration of an example system for optimizing the assignments of providers to exams and provider workflows;

FIG. 2 is an illustration of an example method for assigning providers to exams;

FIG. 3 is an illustration of an example method for simulating a provider workflow;

FIG. 4 is an illustration of an example method for assigning exams;

FIG. 5 is an illustration of an example method for simulating a provider workflow; and

FIG. 6 shows an example computing environment in which example embodiments and aspects may be implemented.

### DETAILED DESCRIPTION

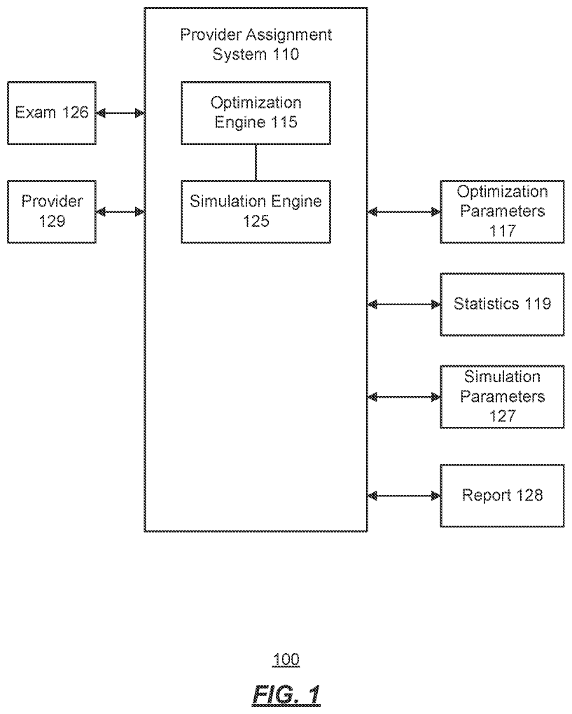

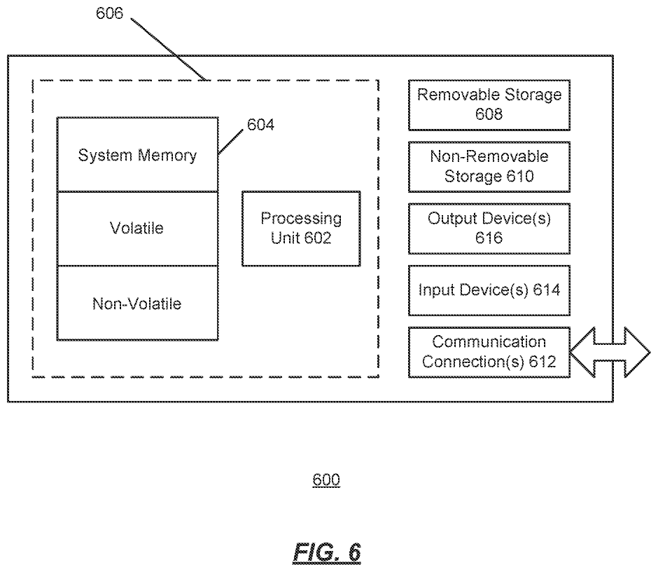

FIG. 1 is an illustration of a provider assignment system 110 that assigns providers 129 to exams 126. The provider assignment system 110 may be implemented using one or more general purpose computing devices such as the computing device 600 illustrated in FIG. 6.

Exams 126 (also referred to as tasks) may include a variety of medical exams that can be completed by a provider 129 and include exams 126 such as reading an X-ray, CT scan, ultrasound, or MRI. Other types of exams may be supported. Each exam 126 may be associated with metadata that may be used by the provider assignment system 110 to assign a provider 129 to the exam 126. The metadata may include a service level agreement (“SLA”) describing how fast the exam 126 should be completed, a difficulty associated with reading the exam 126, an urgency associated with the exam 126, an indication of the specialties or experience level of the provider 129 that should read the exam 126, and a location of the patient associated with the exam 126 (e.g., the entity, hospital, or medical facility where the patient is located or where the exam was performed). Other metadata may be associated with each exam 126.

The providers 129 may be physicians, technicians, nurses or other healthcare providers that are part of a pool that is available to read or complete exams 126 that are assigned to them. Depending on the embodiment, as exams 126 are received by the provider assignment system 110, available providers 129 are assigned the exams 126 by the provider assignment system 110 using the optimization engine 115. The providers 129 may then complete the exams 126 in the order that they are assigned to them. This process is referred to herein as the provider workflow.

Similar to the exams 126, each provider 129 may be associated with metadata that may be used by the provider assignment system 110 to assign providers 129 to exams 126. The metadata may include information such as the specialty or specialties associated with each provider, the type(s) of exams 126 preferred by each provider 129, an indication of how fast or quickly the provider 129 can work, where the provider 129 is located, a current workload of the provider 129, and an indication of the overall quality of the provider 129. Quality may refer to scores given to a provider 129 by other providers 129 or patients, for example. Other quality measures may be used.

As exams 126 are ordered and are received by the provider assignment system 110, the provider assignment system 110 may use the optimization engine 115 to assign a provider 129 of the available providers 129 to the exams 126. The optimization engine 115 may, for a provider 129, select the optimal exam 126 to assign to the provider 129 based on some combination of optimization parameters 117 selected by a user or administrator and the metadata associated with each exam 126 and provider 129.

Depending on the embodiment, the optimization parameters 117 may include, for example, quality (e.g., assign to the provider 129 best suited to read or complete the exam 126), speed (e.g., assign such that exams 126 are completed in the fastest amount of time or within an associated SLA), load-balancing (e.g., assign such that each provider 129 has approximately the same amount of work), location (e.g., assign a provider 129 that has a same location as the exam 126), and provider preferences (e.g., assign the provider 129 that most prefers that type of exam 126). Other optimization parameters 117 may be supported.

In some embodiments, the optimization parameters 117 may further include one or more constraints. As used herein a constrain may include any parameters that constrain or limit the number of exams that may be performed by an entity (e.g., hospital, clinic, or telemedicine provider).

One example of a constraint is referred to herein as a budget constrain. For the budget constraint, each exam 126 may be assigned a cost. The cost may be fixed or may be dependent on the type of the exam 126. The budget constraint may indicate a maximum amount of money that can be spent by the entity over a given time period. Accordingly, when using the budget constrain for a period, the optimization engine 115 may only assign providers 129 to a number of exams 126 whose total cost does not exceed the budget.

Another example constraint is a quota constraint. Each provider 129 may be assigned a fixed quota or number of exams 126 that may be completed by the provider 129 for a period. The optimization engine 115 may assign exams 126 to providers 129 so that no provider 129 exceeds their quota for the period. The quota for each provider 129 may be set by the entity.

An administrator of the entity may provide the optimization engine 115 with the values for the optimization parameters 117. For example, the administrator may use a user interface to specify what weight the optimization engine 115 should use for each of the specified optimization parameters 117. If the administrator wants the optimization engine 115 to consider only quality when assigning providers 129 to exams 126, the optimization engine 115 may set the weight for the quality parameter to 1 and the weights for all other parameters to 0. If the administrator wants the optimization engine 115 to consider quality and speed equally when assigning providers 129 to exams 126, the optimization engine 115 may set the weight of the quality parameter to 0.5 and the weight of the speed parameter to 0.5, and all other parameters to 0. If the administrator wants the optimization engine 115 to consider quality and speed equally when assigning providers 129 to exams 126, but also consider provider preferences to some extent, the optimization engine 115 may set the weight of the quality parameter to 0.4, the weight of the speed parameter to 0.4, the weight of the provider preference to 0.2, and all other parameters to 0.

Once the optimization parameters 117 have been selected or defined, the optimization engine 115 may assign providers 129 to exams 126 using the metadata associated with the exams 126, the metadata associated with each provider 129, and the optimization parameters 117 and their respective weights. The exams may be selected from a backlog where received exams 126 wait to be completed by a provider 129.

In some embodiments, the provider assignment system 110 may assign providers 129 to exams 126 by solving the optimization problem according to the specified optimization parameters 117 for all exams 126 in the backlog and all available providers 129. Any method for solving an optimization problem may be used. Once an optimal exam 126 has been selected for each available provider 129, the exams 126 may be assigned.

The provider assignment system 110 may generate and record a variety of statistics 119 regarding the exams 126 and providers 129. These statistics 119 may include statistics about each provider 129, such as the number of exams 126 handled, and the rate at which the provider 129 handles each type of exam 126. The statistics 119 may further include, for example, the total number of exams 126 waiting to be handled by the provider 129, the total number of providers 129 that are available to handle exams 126, and the number of each type of exam 126 that has been received. The statistics 119 may include historical data about each exam 126 and provider 129 associated with the provider assignment system 110.

The optimization engine 115 may further estimate the expected availability of each provider 129 for a period. The expected availability for a provider 129 may be used by the optimization engine 115 to determine when a particular provider 129 will become available to handle a new exam 126 and to determine how long the provider 129 will complete a current exam 126.

In some embodiments, the optimization engine 115 may estimate the expected availability of a particular provider 129 for a period based on the historical data about the provider 129 at or around the same period in the statistics 119. The historical data for a provider 129 for a period may indicate the number of exams 126 that the provider 129 handled during the period, the total amount of time the provider 129 was available to work during the period, and how long the provider 129 took to complete each exam 126. Other information may be included in the historical data.

In some embodiments, optimization engine 1115 may estimate the availability for a provider 129 using a model. The model may take as an input an indicator of a provider 129 and a period and may output the expected availability of the provider 129 for the period. The model may be a machine learning model or a neural network trained using the historical data from the statistics 119. Any method for training a model may be used.

The optimization engine 115 may continuously monitor changes to the exams 126 and providers 129, and the statistics 119, and may make changes to some or all of the assignments in response to the changes and/or statistics 119. For example, if the number of providers 129 that are available to handle the pending exams 126 changes, the optimization engine 115 may re-determine the optimal assignment for each provider 129 in view of the change in the number of providers 129 (including their associated metadata). In another example, as exams 126 are completed by providers and new exams 126 with different metadata are received, the optimization engine 115 may re-determine the optimal assignments for the providers 129. Depending on the embodiment, the optimization engine 115 may re-assign the providers 129 and/or exams 126 in response to changes at scheduled times, or in response to an instruction from an administrator.

The optimization engine 115 may further reoptimize the exam 126 and provider 129 assignments in view of changes to the optimization parameters 117 and/or their respective weights. For example, the administrator may initially use the optimization parameters of 0.9 speed and 0.1 quality. However, the administrator may use the statistics 119 to determine that the system has a low number of pending exams 126, and therefore may change the optimization parameters 117 to 100% quality. In response, the optimization engine 115 may re-assign the providers 129 to the pending exams 126 based on the new optimization parameters 117.

The provider assignment system 110 may further include a simulation engine 125 that may allow the administrator to run one or more simulations to test how various changes to the optimization parameters 117 and/or other variables, such as changes to the number of available providers 129 or the volume of exams 126 that are received, may affect the operation of the provider assignment system 110 and the provider workflows.

In one embodiment, the simulation engine 125 may run the simulations by first specifying simulation parameters 127. The simulation parameters 127 may include a time frame for the simulation (e.g., one week, one month, one year), the particular optimization parameters 117 that should be used by the optimization engine 115 during the simulation, the number and types of exams 126 that should be received during the simulation, and the number and types of the providers 129 that should be available during the simulation. In addition, the simulation parameters 127 may specify a particular date in the past, such that the simulation engine 125 may then use the statistics 119 and historical data associated with the particular date to determine the number of exams 126 that will be received and the number of providers 129 that may be available.

The simulation engine 125 may then run a simulation according to the simulation parameters 127. In particular, the simulation engine 125 may run the simulation by providing exams 126 and providers 129 to the optimization engine 115 according to the simulation parameters 127 and/or the statistics 119. The optimization engine 115 may then assign the (fake) exams 126 to the (fake) providers 129 without knowing that a simulation is taking place. The simulation engine 125 may fake the completion of each exam 126 according to the metadata associated with the assigned provider 129. The simulation engine 125 may simulate how long each exam 126 took to complete based on statistics 119 collected about each type of exam 126 and provider 129.

After running the simulation, the simulation engine 125 may generate a report 128 that includes several key performance indicators measured during the simulation. These may include, for example, how long each exam 126 took to complete, whether the SLAs associated with each exam 126 were met or exceeded, how busy each provider 129 was, what the total costs in provider 129 time, etc. were. Other key performance indicators may be included in the report 128.

As may be appreciated, the simulation engine 125 allows an administrator to determine how proposed changes to the provider assignment system 110 will affect key performance indicators. For example, an administrator may desire to determine how the key performance indicators will be affected if a certain number or type of providers 129 are added or removed from the available providers 129, if the number of exams 126 increases by twenty percent due to a pandemic, if the number of providers 129 at a particular location is changed, and/or if particular optimization parameters 117 are adjusted. The administrator may further use the simulation engine 125 to determine optimal numbers of providers 129 and optimization parameters 117 by simulating the provider workflow for a variety of different simulation parameters 127 and comparing the results from the reports 128.

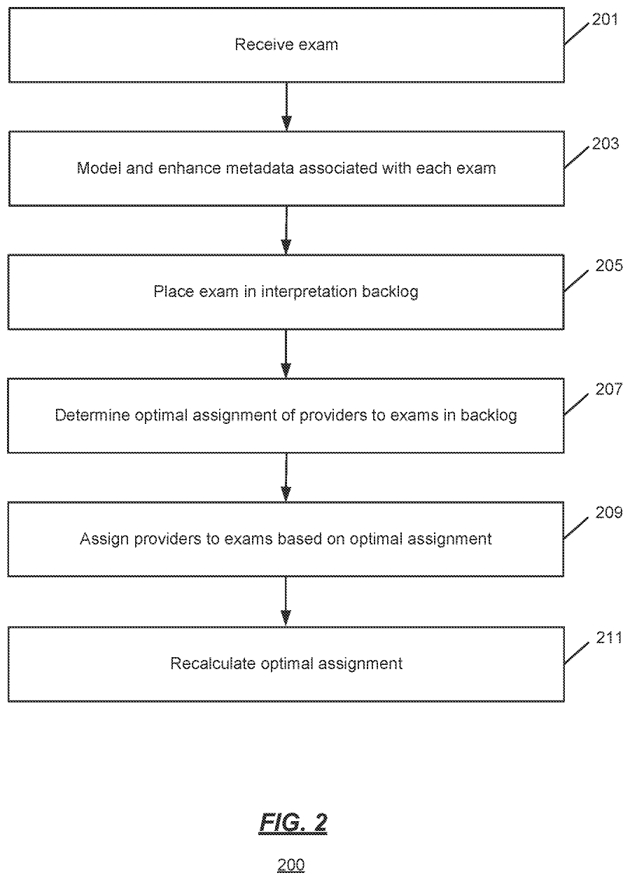

FIG. 2 is an illustration of an example method for assigning providers to exams. The method 200 may be implemented by the provider assignment system 110.

At 201, an exam is received by the provider assignment system 110.

At 203, the metadata associated with the exam 126 is modeled and enhanced to determine the priority of the exam 126. The priority may be determined using the optimization engine 115 according to the optimization parameters 117.

At 205, the exam is placed into an interpretation backlog. The interpretation backlog may be a queue where exams 126 wait to be assigned to providers 120.

At 207, the optimal assignment of the providers to exams from the backlog is determined. The optimal assignment may be determined by the optimization engine 115 using the optimization parameters 117. In some embodiments, the optimization engine 115 may further estimate the availability of each provider 129 and may consider the availability of each provider 129 when determining the optimal assignment. The optimization engine 115 may estimate the availability of a provider using historical data associated with the provider 120 and/or a prediction model trained using the historical data.

At 209, the providers 129 are assigned to exams according to the optimal assignment. The exams 126 may be assigned by the optimization engine 115.

At 211, after each exam 126 is completed (or any other changes are detected such as a change in the number of providers 129), the optimal assignment of providers 129 to exams 126 is recalculated by the optimization engine 115.

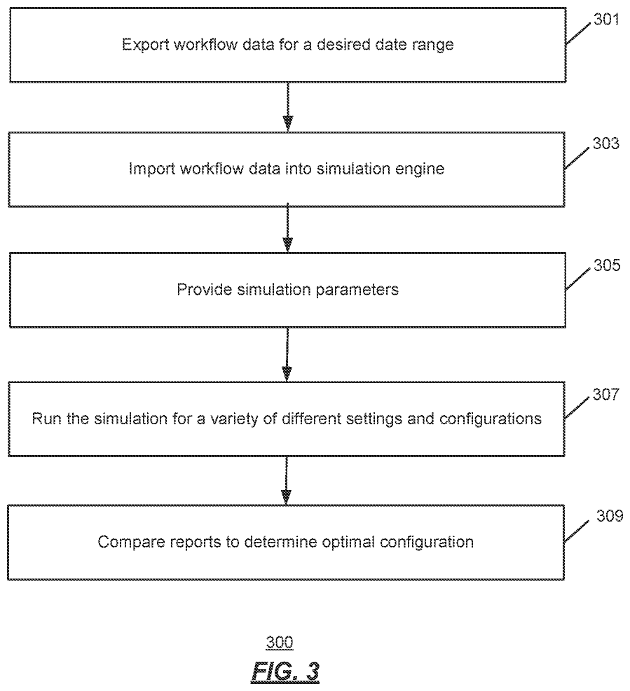

FIG. 3 is an illustration of an example method for simulating a provider workflow. The method 300 may be implemented by the provider assignment system 110.

At 301, workflow data is exported for a desired date range. The workflow data may be part of the statistics 119 and may be exported by the simulation engine 125 based on simulation parameters 127 provided by an administrator.

At 303, the workflow data is imported by the simulation engine 125.

At 305, the simulation parameters 127 are provided by the administrator. The simulation parameters 127 may include changes to the number of providers 129, locations of the providers 129, and qualifications and/or specialties of the providers 129. The simulation parameters 127 may further specify changes to the optimization parameters 117 to use during the simulation.

At 307, the simulation is run for a variety of different configurations. Each configuration may correspond to a different set of simulation parameters 127. The simulations may be run for the same time periods by the simulation engine 125.

At 309, the reports 128 for each simulation are compared to determine the combination of providers 129 and optimization parameters 117 that resulted in the best KPIs.

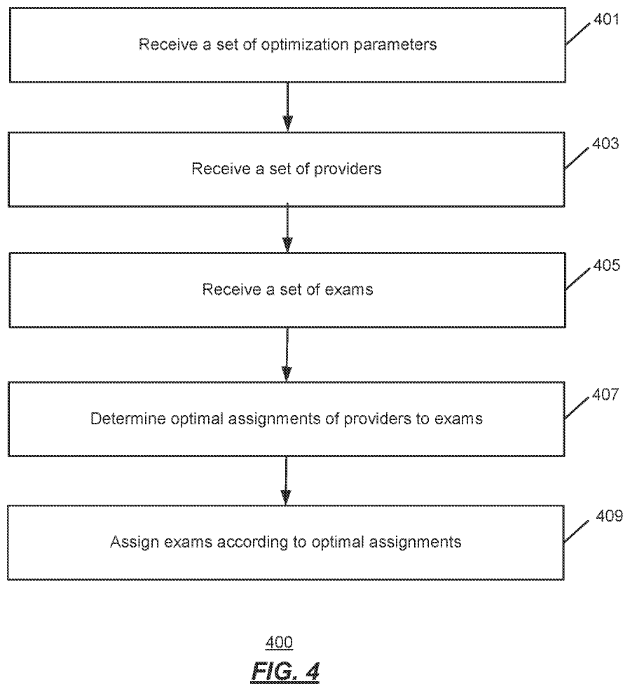

FIG. 4 is an illustration of an example method for assigning exams according to one embodiment. The method 400 may be implemented by the provider assignment system 110.

At 401, a set of optimization parameters is received. The optimization parameters 117 may be received by the optimization engine 115 of the provider assignment system 110. The parameters 117 may be received from an entity (e.g., hospital or clinic) and may include quality, speed, load-balancing, location, and provider preferences. In some embodiments, the parameters may further include one or more constraints such as budget and construction constraints.

At 403, a set of providers is received. The set of providers 129 may be identifiers of providers 129 and may be received by the optimization engine 115. The providers 129 may be providers 129 that are available to work during a period or at a particular time. Each identified provider 129 may be associated with metadata. The metadata may include information such as the specialty or specialties associated with each provider, the type(s) of exams 126 preferred by each provider 129, an indication of how fast or quickly the provider 129 can work, where the provider 129 is located, a current workload of the provider 129, and an indication of the overall quality of the provider 129. Other metadata may be included.

At 405, a set of exams is received. The set of exams 126 may be identifiers of exams 126 that have received from the entity. Depending on the embodiment, the exams 126 may include one or more medical exams or procedures. Each identified exam 126 may be associated with metadata. The metadata may include an SLA, a difficulty, an urgency or time frame for completing the exam, an indication of the specialties or experience level of the provider 129 that should read or handle the exam, and a location of the patient associated with the exam 126. Other metadata may be included.

At 407, an optimal assignment of providers to exams is determined. The optimal assignment may be determined by the optimization engine 115 using the metadata associated with each provider and each exam. Depending on the embodiment, the optimization engine 115 may also consider one or more constraints when determining the optimal assignment.

In some embodiments, the optimization engine 115 may further estimate the availability of each provider 129 using a model that is trained based on historical data about the availability of the providers 129. The optimization engine 115 may consider the estimated availability of each provider 129 when determining the optimal assignment.

At 409, the exams are assigned according to the optimal assignments. The exams 126 may be assigned to providers 129 by the optimization engine 115. As providers 129 finish exams 126, providers 129 become unavailable, new providers 129 become available, and new exams 126 are received, the optimization engine 115 may recalculate the optimal assignment.

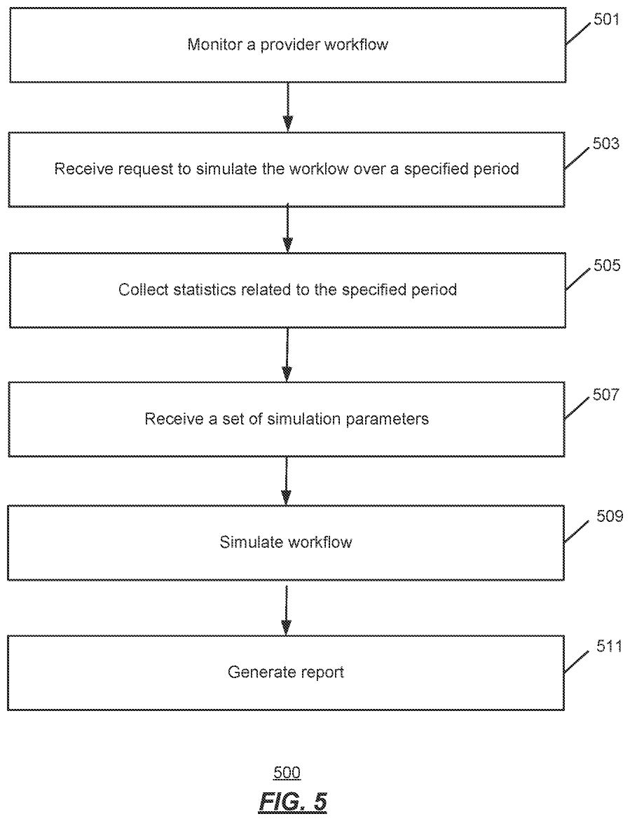

FIG. 5 is an illustration of an example method for simulating a provider workflow. The method 500 may be implemented by the provider assignment system 110.

At 501, a provider workflow is monitored. The provider workflow may be monitored by the simulation engine 125 over a plurality of periods to generate statistics 119 for each period. The statistics 119 may include statistics about the providers 129 that worked during the period and the exams 126 that were handled during the period. In particular, the statistics 119 may include historical data about each provider 129 and exam 126 associated with the period. The statistics 119 may also include various KPIs about the workflow for the period. The KPIs may include average patient wait time, average turn-around time for exams 126, and patient satisfaction scores or reviews. Other performance indicators may be included in the statistics 119.

At 503, a request to simulate the provider workflow over a specified period is received. The request may be received by the simulation engine 125 and the specified period may be one of the periods that the provider workflow was monitored at 501.

At 505, statistics related to the specified period are collected. The statistics 119 may be collected by the simulation engine 115 from among the statistics 119 that were generated from the workflow monitoring for the specified period.

At 507, a set of simulation parameters is received. The set of simulation parameters 127 may be received by the simulation engine 125 as part of the simulation request. The simulation parameters 127 may include changes to the provider workflow for the period such changing the number of exams 126, the types of exams 126, the number of providers 129 that worked during the specified period, and the specialties or experience levels of the providers 129. For example, an administrator may desire to see how KPIs such as average wait time or average handling time for exams 126 is affected by a ten percent reduction in the number of providers 129. As another example, the administrator may wish to stress test the provider workflow by increasing the number of exams 126 that were received during the period by ten percent.

At 509, the workflow is simulated, the workflow may be simulated by the simulation engine 125 using the collected statistics 119 and the simulation parameters 127. In some embodiments, the simulation engine 125 may run the simulation by providing exams 126 and providers 129 to the optimization engine 115 according to the simulation parameters 127 and/or the statistics 119. The optimization engine 115 may then determine the optimal assignment of exams 126 to providers 129 as described above. The simulation engine 125 may simulate the completion of exams 126 by providers 129 and may add or remove providers 129 from the available providers 129 according to the statistics 119.

At 511, a report is generated. The report 128 may be generated by the simulation engine 125 based on the simulation of the workflow. The report 128 may include the simulation parameters 127 and one or more KPIs measured during the simulation.

FIG. 6 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, cloud-based systems, microprocessor-based systems, network personal computers (PCs), minicomputers, mainframe computers, embedded systems, distributed computing environments that include any of the above systems or devices, and the like. The computing environment may include a cloud-based computing environment.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 6, an example system for implementing aspects described herein includes a computing device, such as computing device 600. In its most basic configuration, computing device 600 typically includes at least one processing unit 602 and memory 604. Depending on the exact configuration and type of computing device, memory 604 may be volatile (such as random access memory (RAM)), non-volatile (such as read-only memory (ROM), flash memory, etc.), or some combination of the two. This most basic configuration is illustrated in FIG. 6 by dashed line 606.

Computing device 600 may have additional features/functionality. For example, computing device 600 may include additional storage (removable and/or non-removable) including, but not limited to, magnetic or optical disks or tape. Such additional storage is illustrated in FIG. 6 by removable storage 608 and non-removable storage 610.

Computing device 600 typically includes a variety of computer readable media. Computer readable media can be any available media that can be accessed by the device 600 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 604, removable storage 608, and non-removable storage 610 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by computing device 600. Any such computer storage media may be part of computing device 600.

Computing device 600 may contain communication connection(s) 612 that allow the device to communicate with other devices. Computing device 600 may also have input device(s) 614 such as a keyboard, mouse, pen, voice input device, touch input device, etc. Output device(s) 616 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A system for assigning exams to providers comprising:
at least one computing device; and
a memory storing computer-readable instructions that when executed by the at least one computing device cause the at least one computing device to:
receive a set of optimization parameters;
receive a set of providers, wherein each provider is associated with metadata;
receive a set of exams, wherein each exam is associated with metadata;
determine an optimal assignment of providers to exams based on the optimization parameters, the metadata associated with each exam, and the metadata associated with each provider; and
provide at least some of the exams to the providers according to the optimal assignment.

2. The system of claim 1, further comprising computer-readable instructions that when executed by the at least one computing device cause the at least one computing device to:
receive an indication of a change, and in response to the indication redetermine the optimal assignment.

3. The system of claim 2, wherein the change is a change to one or more of a number of providers in the set of providers, one or more of the optimization parameters, the completion of an exam of the set of exams, and the receipt of a new exam.

4. The system of claim 1, wherein the optimization parameters comprise one or more of speed, quality, provider preference, and location.

5. The system of claim 1, wherein the optimization parameters comprise one or more constraints.

6. The system of claim 5, wherein the one or more constraints comprise budget constraints or production constraints.

7. The system of claim 1, further comprising computer-readable instructions that when executed by the at least one computing device cause the at least one computing device to: estimate an expected availability of each provider of the set of providers, and determine the optimal assignment of providers to exams based on the optimization parameters, the metadata associated with each exam, the metadata associated with each provider, and the expected availability of each provider of the set of providers.

8. The system of claim 7, further comprising computer-readable instructions that when executed by the at least one computing device cause the at least one computing device to:
for each provider of the set of the providers, retrieve historical data about each exam of the set of exams for the provider; and
for each provider of the set of providers, estimate the expected availability of the provider using the retrieved historical data.

9. The system of claim 7, further comprising computer-readable instructions that when executed by the at least one computing device cause the at least one computing device to:
for each provider of the set of the providers, retrieve historical data about each exam of the set of exams for the provider;
train a model using the retrieved historical data for each provider; and
estimate the expected availability of each provider using the model.

10. A method for simulating a provider workflow:
monitoring a provider workflow to generate a plurality of statistics related to a plurality of providers, a plurality of exams, and a plurality of optimization parameters associated with the workflow by a computing device;
receiving a request to simulate the workflow over a specified period by the computing device;
collecting the plurality of statistics corresponding to the specified period by the computing device;
receiving a set of simulation parameters by the computing device, wherein the simulation parameters include one or more of changes to a number of providers associated with the workflow during the duration, changes to metadata associated with the providers, and changes to the optimization parameters during the workflow;
simulating the workflow during the specified period using the plurality of statistics and the set of simulation parameters by the computing device; and
generating a report based on the simulation by the computing device, wherein the report comprises a plurality of key performance Indicators.

11. The method of claim 10, further comprising:
changing one or more of the simulation parameters;
simulating the workflow using the changed simulation parameters;
generating another report based on the new simulation; and
comparing the plurality of Key Performance Indicators in each report to determine one or more optimal optimization parameters.

12. The method of claim 10, further comprising:
changing one or more providers of the plurality of providers;
simulating the workflow using the changed one or more providers;
generating another report based on the new simulation; and
comparing the plurality of Key Performance Indicators in each report to determine an optimal plurality of providers.

13. A method comprising:
receiving at least one optimization parameter by a computing device;
receiving a set of providers by the computing device, wherein each provider is associated with metadata;
receiving an exam by the computing device, wherein the exam is associated with metadata;
estimating an expected availability of each provider of the set of providers by the computing device;
determining an optimal assignment of a selected provider of the set of providers to the exam based on the optimization parameter, the metadata associated with the exam, and the expected availability of the selected provider, and the metadata associated with the selected provider by the computing device; and
providing the exam to the selected provider according to the optimal assignment by the computing device.

14. The method of claim 13, wherein estimating the expected availability of each provider of the set of providers comprises:
for each provider of the set of the providers, retrieving historical data about the exam for the provider; and
for each provider of the set of providers, estimating the expected availability of the provider using the retrieved historical data.

15. The method of claim 13, wherein estimating the expected availability of each provider of the set of providers comprises:
for each provider of the set of the providers, retrieving historical data about the exam for the provider;
training a model using the retrieved historical data for each provider; and
estimating the expected availability of each provider using the model.

16. The method of claim 13, further comprising receiving an indication of a change, and in response to the indication redetermining the optimal assignment.

17. The method of claim 16, wherein the change is a change to one or more of a number of providers in the set of providers, one or more of the optimization parameters, the completion of an exam of the set of exams, and the receipt of a new exam.

18. The method of claim 13, wherein the optimization parameters comprise one or more of speed, quality, provider preference, and location.

19. The method of claim 13, wherein the optimization parameters comprise one or more constraints.

20. The method of claim 19, wherein the one or more constrains comprise budget constraints or production constraints.
